(19) United States
(12) Patent Application Publication
(10) Pub. No.: US 2024/0399164 A1
(43) Pub. Date: Dec. 5, 2024

(54) Multi-function Device For Treating Skin

(71) Applicant: SOLAWAVE INC., Los Angeles, CA (US)

(72) Inventors: Andrew Silberstein, Los Angeles, CA (US); Eli Bailey, Los Angeles, CA (US); Michael Kasbergen, The Hague (NL); Christopher Buettner, Woodbridge, VA (US)

(21) Appl. No.: 18/200,473

(22) Filed: May 22, 2023

(51) Int. Cl. A61N 5/06; A61F 7/00; A61H 23/02; A61N 1/32

(52) U.S. Cl. CPC A61N 5/0616; A61F 7/007; A61H 23/02; A61N 1/328; A61F 2007/006; A61F 2007/0075; A61F 2007/0086; A61F 2007/0096; A61H 2201/0207; A61H 2201/0214; A61H 2201/0221; A61H 2201/025; A61H 2201/0257; A61H 2201/0285; A61H 2201/10; A61H 2201/5038; A61H 2201/5082; A61N 2005/0663

(57) ABSTRACT

A skin treatment device that includes a housing, a battery, a controller, connected to the battery that includes a processor; and a memory in communication with the processor for storing program instructions and data, a red and a blue LED, a set of user-selectable controls, a right and a left Peltier element, each with a bottom side that contacts a corresponding treatment pad, where during operational use the treatment pad contacts the skin of a user to deliver heating or cooling, or microcurrent, and wherein the instructions are operable to prevent either of the bottom sides of the right and left thermal elements from exceeding a maximum temperature and also to maintain the temperature of the bottom sides of the two thermal elements within a temperature threshold value.

### BACKGROUND OF THE INVENTION

### Field of the Invention

This invention relates to handheld medical devices that treat skin conditions.

### Description of the Related Art

There are a variety of consumer handheld devices that treat skin conditions. These include: light therapy, microcurrent, hot and cold treatments, and vibration or massage therapy. Each of these treatments are applied by specialists such as dermatologists and estheticians; but increasingly, consumer devices for use in the home are available to apply these therapies.

For example, light therapy is used to treat a range of skin concerns such as acne, eczema, wrinkles, and psoriasis. A growing number of consumer devices that can be used in the home are commercially available.

Microcurrent devices use low-voltage electricity to stimulate muscle, adenosine triphosphate (ATP) cell growth, and collagen development in the dermis of the face. Generally, the objective is to tighten the skin to improve appearance.

Hot and cold treatments are used to achieve stronger, healthier skin. In a hot/cold treatment, typically heat is applied to the skin to help increase blood flow and circulation. The heat treatment is typically followed by a cold treatment that helps reduce any inflammation or swelling in the skin. Hot and cold treatments are also used to improve the absorption and retention of topically applied cosmetics.

For a consumer, the choice of the right device for skin treatment can be confusing. It can be expensive to purchase multiple devices, performing a single treatment. Thus, a single, multi-function device that can apply any of the popular treatments is desirable. Further, it is desirable to be able to apply sequences of treatments, while respecting user preferences and settings.

### SUMMARY OF THE INVENTION

The subject invention is a hand-held consumer medical device, referred to herein as a skin treatment device or simply device, which provides several skin treatments, including light therapy, micro-current, heating and cooling, and vibration. It combines the function of multiple devices into a single, compact, consumer device. The device includes an easy-to-use user interface with controls for each of the different treatment types.

The invention provides two surfaces, referred to as treatment pads, each of which contacts a user's skin and is capable of providing heating and cooling, micro-current, and vibration. In addition, an integrated light therapy capability is provided.

Each treatment pad has a corresponding thermal element that generates heating or cooling, through the treatment pad. An innovative control method and associated circuitry synchronizes the relative temperatures of the two treatment pads to ensure that they are nearly identical at all times, regardless of whether one or both treatments pads is in contact with the user's skin.

Certain embodiments relate to a skin treatment device that includes a housing, a power source, a controller, connected to the power source that includes a processor; and a memory in communication with the processor for storing program instructions and data, a red or a blue light, operable to emit red, blue or mixed red and blue light onto the skin of the user, a set of user-selectable controls, the controls including an on/off control, a hot/cold control, a microcurrent control, and a light therapy control that controls the current supplied to the LEDs, a right and a left thermal element, each with a bottom side that contacts a treatment pad, where during operational use the treatment pad contacts the skin of a user to deliver heating or cooling, or microcurrent, and wherein the instructions are operable to implement an algorithm that controls the right and left thermal elements to maintain the temperature of the bottom sides of the two thermal elements within a temperature threshold value.

In certain embodiments, the skin treatment device further includes a right and a left absorption rod, wherein the right absorption rod attaches at one end to the right thermal element and at the other end to a right heat sink, and the left absorption rod attaches at one end to the left thermal element and at the other end to a left heat sink; a right fan above the right heat sink and a left fan above the left heat sink rod; and an air passageway between the right heat sink and the right fan and a left air passageway between the left heat sink and the left fan, each air passageway enabling air to flow from the fan to a vent in the housing that enables air to flow to the exterior of the device wherein the air passageway further enables heat transferred by the right and left thermal element to be transferred by a respective absorption rod through the heatsink to the air flowing through the air passageway, a right and a left thermal plate on the surface of the device, the right thermal plate positioned above the right fan and the left thermal plate positioned above the left fan, where both the left and right thermal plates include one or more vents, and where during operation of the device, the fan draws air from the exterior into the air passageway.

Other embodiments relate to a processor-implemented method performed by a skin treatment device managing the temperatures of two thermal elements, the steps including receiving temperature measurements of a bottom side for each of the two thermal elements, cutting off current to a thermal element if its bottom side reaches a maximum temperature, determining if the difference in temperature between the two thermal elements exceeds a threshold value and upon determining that the difference in temperature between the two thermal elements exceeds a threshold value cutting off the current to the hotter of the two thermal elements.

### BRIEF DESCRIPTION OF DRAWINGS

Non-limiting and non-exhaustive embodiments of the present invention are described with reference to the following drawings. In the drawings, like reference numerals refer to like parts throughout the various figures unless otherwise specified.

FIG. 1 illustrates a first embodiment of the exterior of a skin treatment device that is used to treat the skin of a user.

FIG. 2 illustrates a frontal cross-sectional view of the device.

FIG. 3 illustrates an embodiment of a thermal element ensemble.

FIG. 4A illustrates one embodiment of a control method that manages the temperature of two thermal elements during a heat treatment.

FIG. 4B illustrates one embodiment of a control method 450 that manages the temperatures of two thermal elements 210 during a cooling treatment.

FIGS. 5A and 5B illustrate an embodiment of a printed circuit board (PCB) that controls aspects of the function of the device.

FIG. 6 illustrates a second embodiment of the exterior of a skin treatment device that is used to treat the skin of a user.

FIG. 7 illustrates a frontal cross-sectional view of the skin treatment device of FIG. 6.

FIG. 8 illustrates a second embodiment of a control method that manages the temperatures of two thermal elements during a cooling treatment.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

The invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the invention may be embodied as methods, processes, systems, or devices. The following detailed description is, therefore, not to be taken in a limiting sense.

As used herein the following terms have the meanings given below:

Treatment—as used herein, refers to the use of the skin treatment device by a user on their skin with the objective of obtaining a desired result.

Treatment sequence—as used herein refers to a programmed sequence of different treatment steps, wherein each step is a type of treatment performed for a defined time interval. It is anticipated that the programming and user interface associated with a treatment sequence will be provided by a mobile app or computer application. Generally, the operation of the mobile app and the use of the device to perform treatment sequences is outside the scope of the present disclosure.

### Skin Treatment Device

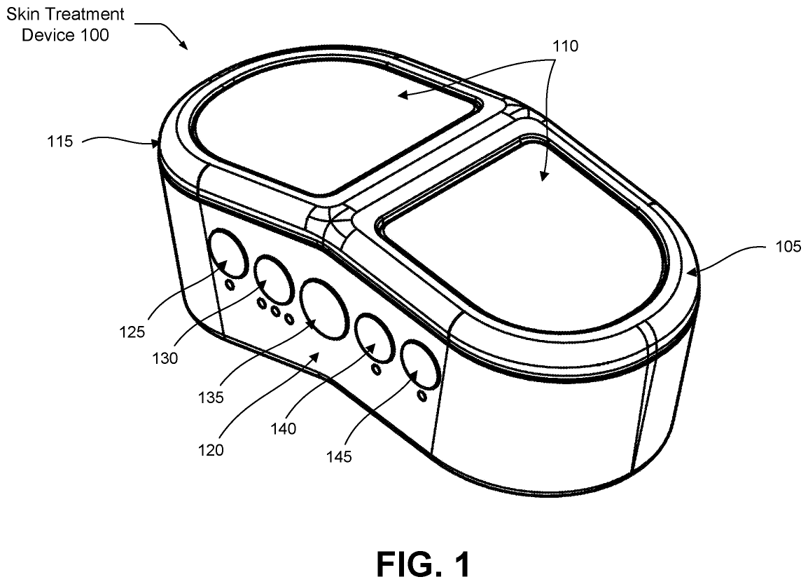

FIG. 1 illustrates an embodiment of the exterior of a skin treatment device 100, henceforth device 100, that is used on the skin. Device 100 has a housing 105 that secures the various elements of the device. Housing 105 may be made of molded plastic or another material such as metal or aluminum.

Generally, the features of device 100 are delivered to a user's skin through one or more pads and a set of lights disposed on the bottom of the device, which is not shown in FIG. 1. Through the plates and the lights, device 100 delivers heating, cooling, an electrical microcurrent charge, vibration, and colored light or light of a specific frequency to the skin.

Device 100 has two thermal plates 110 that dissipate excess heat or cold generated by the device. Most generally, plates 110 are disposed on the surface of device 100. In the embodiment illustrated in FIG. 1 plates 110 are at the top of device 100; however, in other embodiments plates 110 may be disposed on one or more sides of device 100.

Device 100 further has an electrical interface 115 (not shown) that draws an electrical charge suitable for charging a consumer electronic battery. The electrical charge may be used to power device 100 or to charge its battery. Examples of suitable electrical interfaces include inter alia USB, micro USB, USBC, or wireless charging.

Device 100 has a user interface 120 that provides a number of mechanical controls that enable a user to control the device. Additionally, there are one or more indicator lights disposed below each control. The indicator lights indicate the selected value or feature of an associated control. The controls provided by user interface 120 include:

An on/off, or equivalently, start/stop, control 135 enables the user to turn device 100 on or off, i.e. to start and stop operation. During operation, if device 100 is performing a treatment sequence, then control 135 may also act to pause the treatment; in this case, pressing control 135 again causes device 100 to resume the treatment sequence.

A thermal control 125 enables the user to select whether device 100 delivers heating, cooling or none of the above. A color light corresponding to the selected temperature or temperature range may display in proximity to thermal control 125; for example, a red or orange light might display to indicate heating while a blue light might indicate cooling.

A microcurrent control 130 that controls the intensity of the current delivered to the user's skin. Typically, there are several intensity levels, e.g. 0, 1, 2, and 3. These may be activated by sequentially pressing or activating control 130.

A vibration control 140 that controls the intensity of the vibration delivered to the user's skin. In one embodiment, vibration is either off or on. In other embodiments, there are several intensity levels, e.g. 0, 1, 2, and 3. These may be activated by sequentially pressing or activating control 140.

A light therapy control 145 that controls the type of light delivered by device 100. In one embodiment, the light emitted may be red, or blue, or a combination of red and blue such as purple. In other embodiments, the light may be of different colors or if may be electromagnetic radiation at a specific frequency such as infrared or ultraviolet. These may be activated by sequentially pressing or activating control 145 until the desired type of light is emitted.

Pictured below each of controls 125, 130, 140 and 145 are one or more indicator lights. These provide feedback to the user by displaying the status or current selection of the control. For example, the light under control 145 may show the color that is currently selected. Various sizes, numbers, and colors of indicator lights may be used without departing from the scope and spirit of the present invention.

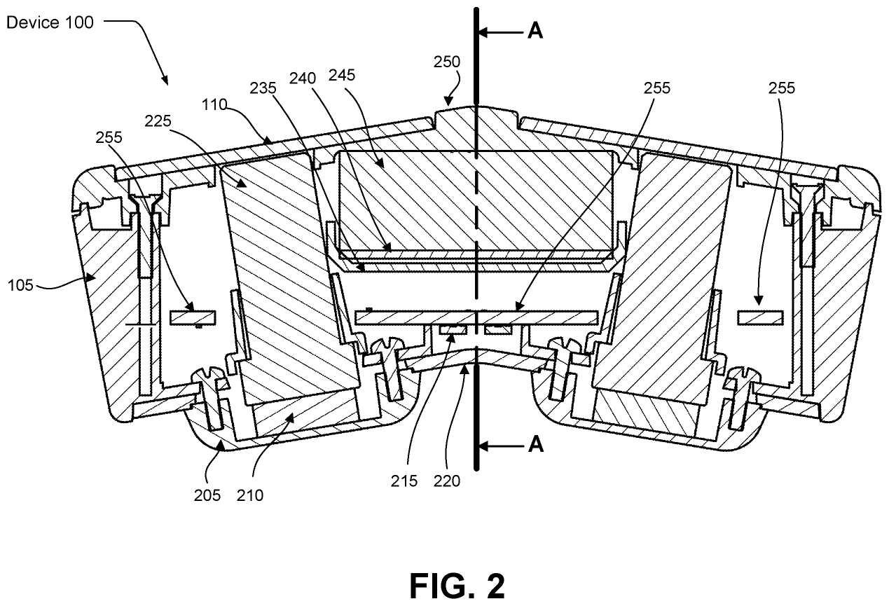

FIG. 2 illustrates a front cross-sectional view of one embodiment of device 100. In this embodiment, device 100 is right-left symmetric about a central vertical axis A. Many of the elements of device 100 are included on both the right side and the left side; accordingly, the function of only one of such pairs is discussed hereinbelow with the understanding that the comments apply equally to each of the two identical instances. As used herein, the term top refers to an upper region that includes right and left thermal elements 210 and a lid 250, while the term bottom refers to a lower region that includes a left and a right treatment pad 205.

Heating and cooling are provided by a thermal element 210. Thermal element 210 transfers heat from one side to the other; which, in this embodiment means transferring heat from the top side of thermal element 210 to its bottom side where the bottom side is in physical contact with or in close physical proximity to treatment pad 205 and the top side is in physical contact with or in close physical proximity to an energy absorption rod 225. In certain embodiments, each thermal element 210 is implemented as a Peltier element that transfers heat using the Peltier effect, which produces a temperature difference between two sides of each element when a current is flowing.

Generally, when the current, supplied by a power source 245, flows in one direction, thermal element 210 supplies heat to energy absorption rod 225. Energy absorption rod 225 transfers the heat to thermal plate 110 which in turn transfers the heat to the surrounding atmosphere. In this embodiment, energy absorption rod 225 either physically contact thermal plate 110 or is in close physical proximity to thermal plate 110 to allow for efficient heat transfer. Concurrently, thermal element 210 supplies cooling to a treatment pad 205. It may be appreciated that in certain embodiments power source 245 is a battery such as a lithium-ion battery that is typically used in consumer electronics devices.

When the current flows in the opposite direction, the side of thermal element 210 in contact with or in close physical proximity to energy absorption rod 225 draws heat, thus cooling energy absorption rod 225. Thermal element 210 then transfers the heat to treatment pad 205.

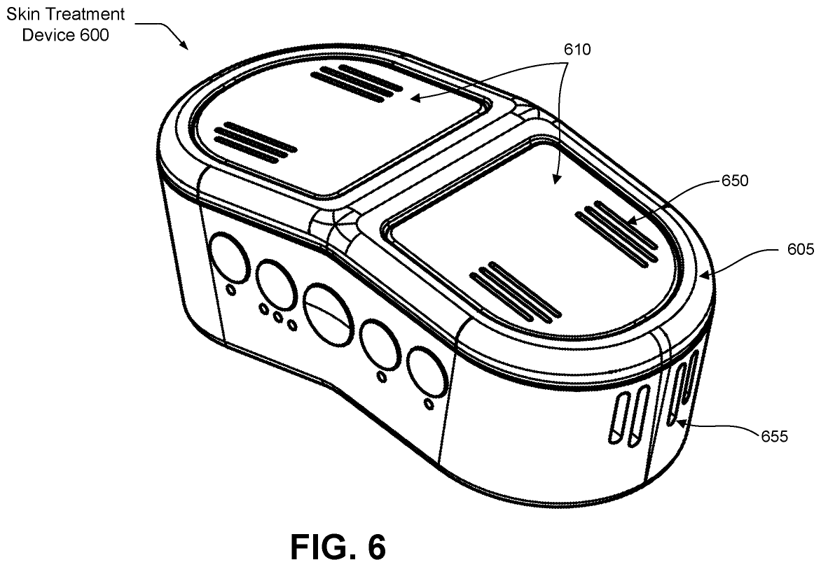

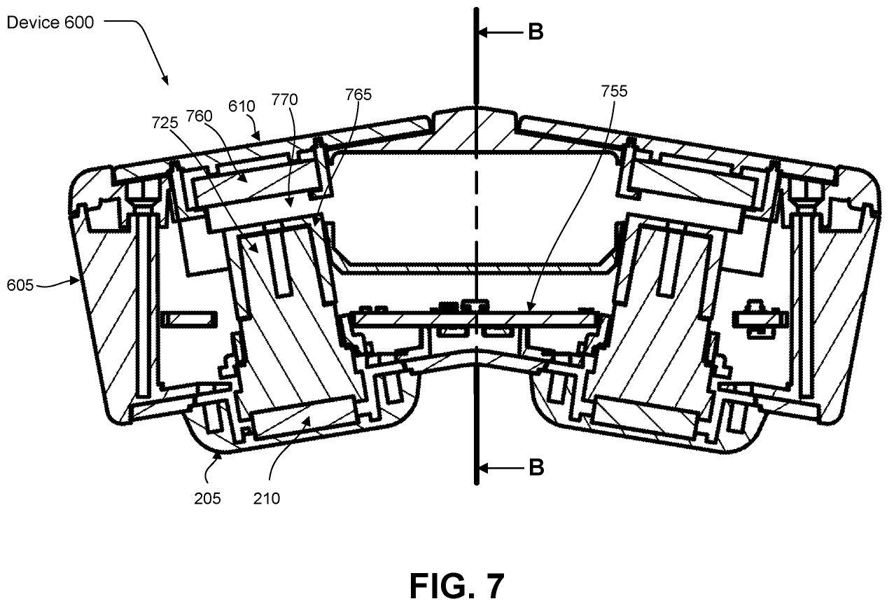

In certain embodiments, thermal element 210 is a commercially available component, such as a Peltier element, that connects to power source 245. For example, a Peltier element can generate up to approximately a 10 degrees Centigrade (C) temperature difference between its two sides when used in a passive configuration, i.e. with no additional ventilation, such as that depicted in FIG. 2. Other embodiments may include additional, stacked, Peltier elements or, alternatively, ventilation may be used to increase the potential temperature difference that can be achieved by device 100. For example, a second embodiment of a skin treatment device that uses a fan to provide ventilation is described below with reference to FIGS. 6-7.

It may be appreciated that other technologies may be used in different embodiments to provide the thermal pump function of thermal element 210 without departing from the scope and spirit of the subject invention. For example, a liquid refrigerant could be used. In yet other embodiments, a heating-only solution that relies, for example, on resistive heating may be employed.

Treatment pad 205 is a thermally conductive covering that conducts the heating or cooling supplied by thermal element 210 to a user's skin. Treatment pad 205 is made of a thermally conductive material such as a metal. In one embodiment, treatment pad 205 is made of stainless steel or aluminum. In certain embodiments, there is a separate treatment pad 205 that corresponds to and covers each thermal element 210. In other embodiments, there is a single treatment pad 205 that covers all thermal elements 210.

At least one treatment light 215 is provided. In certain embodiments, treatment light 215 is implemented by LEDs which generate light with characteristics that have been found to have positive health effects when applied correctly to a user's skin. Typically, the spectral, power and other characteristics follow a 510 (k) predicate submission made and cleared by the U.S. Food and Drug Administration. Such a submission includes a description of a medical device, including all models and accessories or components, as well as device performance specifications. In certain embodiments, light 215 is implemented as one or more LED dies or LED chips, each comprising one or more light emitting LEDs, or lights.

Each light 215 may be of a different color; typically, equal multiples of red and blue lights are provided. For example, lights 215 may comprise two dies for each LED housing, one die emitting red light and one die emitting blue light; and there may be multiple such housings. Treatment lights 215 are activated by light therapy control 145.

Treatment lights 215 are covered by a transparent guard 220. Guard 220 is made of a transparent material such as plastic.

As previously discussed, a right and a left energy absorption rod 225 provides thermal conductivity between treatment pads 205 and thermal plates 110. Absorption rods 225 are made of a thermally conductive material such as metal. In one embodiment, absorption rods 225 are made of cast iron.

Battery holder 235 both secures power source 245 to housing 105 and provides thermal and electrical isolation.

A battery pad 240 provides additional protection for power source 245. Battery pad 240 provides both thermal isolation and shock absorption.

Power source 245 provides current for each of the skin treatments: heating and cooling via thermal elements 210, micro-current, vibration and light therapy, via LEDs 215. In certain embodiments, power source 245 is a rechargeable battery such as a lithium-ion battery typically used in consumer electronic devices.

A lid 250 covers the top of the device 100, specifically covering the power source. Top 250 may be a separate, removeable part or it may be a part of housing 105. Generally, top 250 is part of housing 105, regardless of whether it can be separately removed.

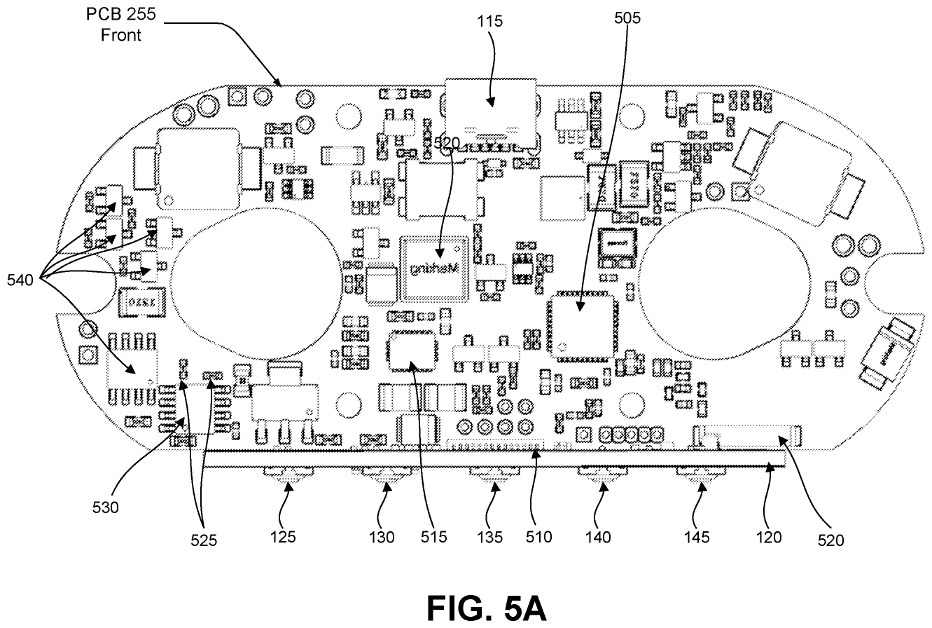

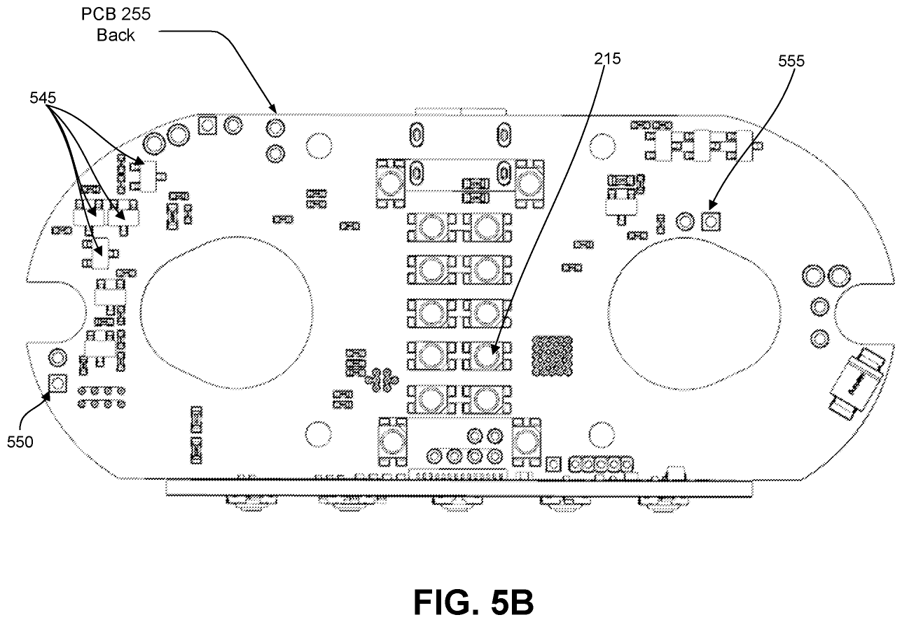

A controller, implemented in certain embodiments as a printed circuit board (PCB) 255, which attaches the main electronic parts, including processor, memory, LED control and temperature synchronization circuitry. It is described in greater detail hereinbelow with reference to FIG. 5.

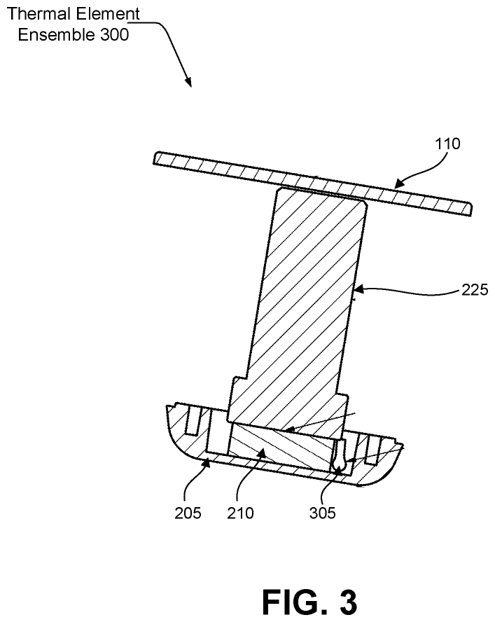

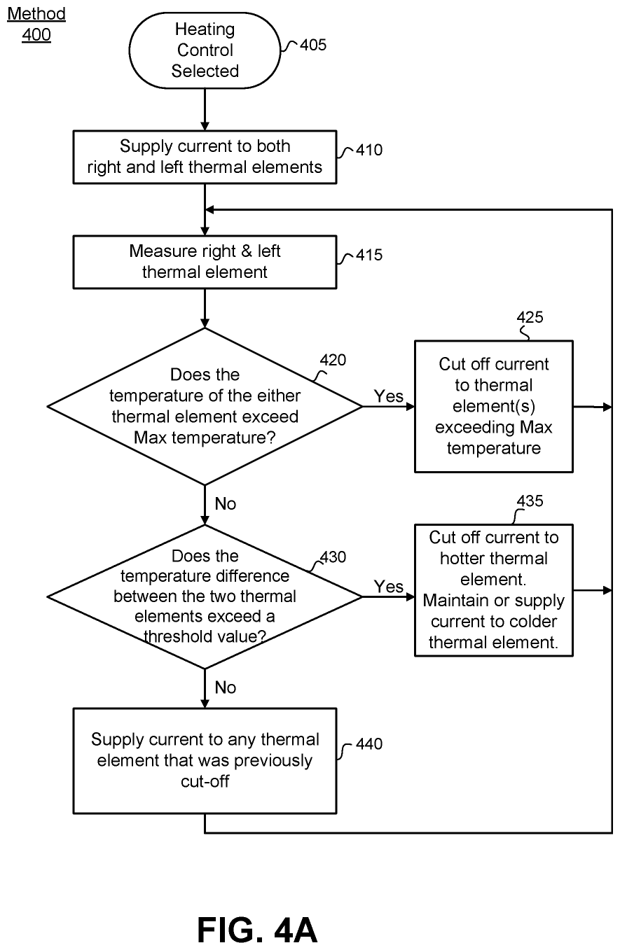

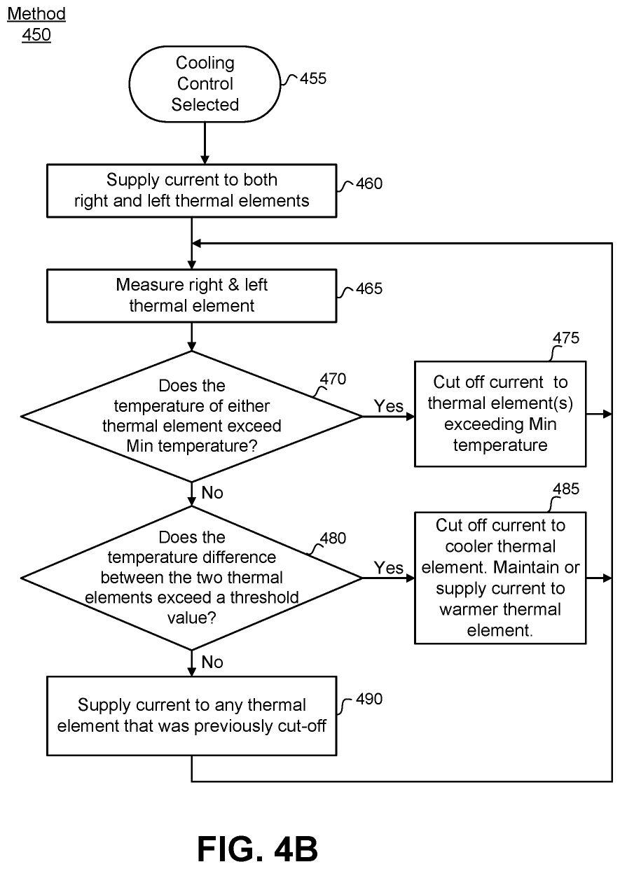

FIG. 3 illustrates an embodiment of a thermal element ensemble 300. There are identical ensembles 300 on the right and left side of device 100. Ensemble 300 includes a thermal plate 110, an energy absorption rod 225, a treatment pad 205, a thermal element 210 and a temperature sensor 305. Temperature sensor 305 measures the temperature of thermal element 210. This is used as input for a temperature synchronization method 400, described in greater detail with reference to FIG. 4.

In one embodiment, thermal plate 110 is made of anodized aluminum. Aluminum is used due to its superior thermal conductivity, enabling it to conduct relative heat or cold from thermal absorption rod 225 and to disperse the heat or cold into the surrounding atmosphere.

### Right-Left Temperature Synchronization

As previously discussed, in certain embodiments device 100 includes both a right and a left thermal element 210, a right and a left treatment pad 205, a right and a left energy absorption rod 225, and a right and a left thermal plate 110. During operation, it is desirable that both right and left treatment pads 205 contact a user's skin and provide heating or cooling at approximately the same temperature. However, a number of factors influence the temperature generated by the two thermal elements 210 on their bottom side, the side that contacts a corresponding treatment pad 205, even when the same current is applied to each. One factor is that due to the manufacturing process there is a variability in the efficiency and variability of thermal elements, for example, when implemented by Peltier elements. Another factor is the potential difference in temperature between the two absorption rods 225 at a particular instant in time. Additionally, in practice it may be the case that only one of the two corresponding treatment pads 205 contacts the user's skin at a particular time. This results in a potential difference in temperature between the right and left treatment pads 205 since only one will disperse heat or cold to the user's skin while the other only contacts the atmosphere, which may be at a significantly different temperature than the user's skin. To counter the potential differences in temperature between the right and left treatment pads 205, a control mechanism is provided that dynamically updates or regulates the current supplied to each of the two thermal elements 210 to maintain the corresponding treatment pads 205 at approximately the same temperature.

FIG. 4A illustrates one embodiment of a control method 400 that manages the temperatures of two thermal elements 210 during a heating treatment. The method includes two sub-methods: (1) steps 420 and 425 together prevent the bottom side of either thermal element 210 from exceeding a designated maximum temperature (Max temperature), and (2) steps 430 and 435 together dynamically turn on and off the current to one or both thermal elements 210 to maintain the temperature difference of their bottom sides, each of which contacts a corresponding treatment pad 205, to within a threshold temperature value. Method 400 applies in the case that the user has selected heat treatment. As will be discussed with reference to FIG. 4B, below, a similar method applies, with only minor changes, in the case that cold treatment is applied.

In method 400, it is assumed that supplying current to a thermal element generates a warmer temperature, up to a point where the thermal element does not get warmer. Further, the phrase “exceeding a Max temperature” means the temperature is hotter than a temperature of Max degrees, where Max is a designated temperature, typically measured in Celsius.

At step 405, method 400 is initiated when a user selects the heating function of device 100.

At step 410 device 100 supplies a preset current to both thermal elements 210. This current may be a single, designated, current, for example, as prescribed by the manufacturer of thermal element 210. Alternatively, in certain embodiments a variable current may be supplied, which varies over time to achieve a desired level of heating or cooling.

At step 415 the temperature of each of the two thermal elements 210 is measured by the corresponding temperature sensor 305.

At step 420 a determination is made as to whether the temperature of either of the two thermal elements 210 exceeds a maximum allowed temperature (Max temperature). This maximum temperature may be established by a medical body or a standards organization or it may be an informal limit. Typically, a maximum temperature of 42 degrees C., established by the IEC 60601 standard is used. IEC 60601 refers to a technical standard from the International Electrotechnical Commission.

At step 425 current is cut off to either or both thermal elements 210 whose temperature is determined to exceed the Max temperature. Then processing returns to step 415.

If at step 420 the temperature of neither thermal element 210 exceeds the Max temperature, then processing continues at step 430.

At step 430 an optional determination is made as to whether the temperature difference between the two thermal elements 210 exceed a designated threshold value, e.g. 1 degree C. The temperatures are within the threshold value if their difference is less than the threshold value. If the temperature difference between the two thermal elements 210 exceeds the designated threshold value, then the method continues at step 435. If the difference in temperatures is within the threshold value, then the method continues at step 440.

At step 435 current is cut off to the hotter of the two thermal elements 210. If current is flowing to the colder thermal element 210 the current is maintained. But if no current is flowing to the colder thermal element 210 then current is turned on to that element. At the conclusion of step 435 processing returns to step 415.

At step 440, if current was previously cut-off to either of the two thermal elements then current is supplied again.

Optionally, a suitable time interval, e.g. 1 second, may be introduced before repeating step 415.

While the above description of method 400 assumes that current is either supplied or not supplied to a thermal element 210, in other embodiments, current may be increased or decreased rather than being entirely shut off, or rather than being supplied only at a fixed, pre-determined, value.

As previously mentioned, method 400 may be applied with minor changes when device 100 is providing cold treatment. FIG. 4B illustrates one embodiment of a control method 450 that manages the temperatures of two thermal elements 210 during a cooling treatment. The method includes two sub-methods: (1) steps 420 and 425 together prevent the bottom side of either thermal element 210 from exceeding a designated minimum temperature (Min temperature), and (2) steps 480 and 485 together dynamically turn on and off the current to one or both thermal elements 210 to maintain the temperature difference of their bottom sides, each of which contacts a corresponding treatment pad 205, to within a threshold temperature value. Method 450 applies in the case that the user has selected cold treatment. In method 450, it is assumed that supplying current to a thermal element generates a colder temperature, up to a point where the thermal element does not get colder. Further, the phrase “exceeding a Min temperature” means the temperature is colder than a temperature of Min degrees, where Min is a designated temperature, typically measured in Celsius.

At step 455, method 450 is initiated when a user selects the cooling function of device 100.

At step 460 device 100 supplies a preset current to both thermal elements 210. This current may be a single, designated, current, for example, as prescribed by the manufacturer of thermal element 210. Alternatively, in certain embodiments a variable current may be supplied, which varies over time to achieve a desired level of heating or cooling.

At step 465 the temperature of each of the two thermal elements 210 is measured by the corresponding temperature sensor 305.

At step 470 a determination is made as to whether the temperature of either of the two thermal elements 210 exceeds a minimum allowed temperature (Min temperature). This minimum temperature may be established by a medical body or a standards organization or it may be an informal limit.

At step 475 current is cut off to either or both thermal elements 210 whose temperature is determined to exceed the Min temperature. Then processing returns to step 465.

If at step 470 the temperature of neither thermal element 210 exceeds the Min temperature, then processing continues at step 480.

At step 480 an optional determination is made as to whether the temperature difference between the two thermal elements 210 exceed a designated threshold value, e.g. 1 degree C. The temperatures are within the threshold value if their difference is less than the threshold value. If the temperature difference between the two thermal elements 210 exceeds the designated threshold value, then the method continues at step 485. If the difference in temperatures is within the threshold value, then the method continues at step 490.

At step 485 current is cut off to the colder of the two thermal elements 210. If current is flowing to the warmer thermal element 210 the current is maintained. But if no current is flowing to the warmer thermal element 210 then current is turned on to that element. At the conclusion of step 485 processing returns to step 465.

At step 490, if current was previously cut-off to either of the two thermal elements then current is supplied again.

While the above description of method 450 assumes that current is either supplied or not supplied to a thermal element 210, in other embodiments, current may be increased or decreased rather than being entirely shut off, or rather than being supplied only at a fixed, pre-determined, value.

FIGS. 5A and 5B illustrate an embodiment of a controller, implemented as a printed circuit board (PCB) 255, which controls aspects of the function of device 100. FIG. 5A illustrates an embodiment of a front side of PCB 255 and FIG. 5B illustrates an embodiment of a back side of PCB 255. In other embodiments, the elements on both sides may be incorporated onto a single side, or onto more than two sides. Notable elements present on PCB 255 include:

Element 505 is a processor assembly, which includes a microprocessor, and static memory used to store program code and data. In other embodiments, the microprocessor and static memory are implemented as two separate elements. Element 505 also includes dynamic memory in certain embodiments. In other embodiments dynamic memory is included as a separate element.

Element 510 provides an interface between buttons 125, 130, 135, 140 and processor assembly 505. Processor assembly 505 activates or deactivates treatments by changing I/O status.

Element 515 converts current from power source 245 as required by PCB 255.

Element 520 is a Bluetooth antenna.

Element 525 represents several circuits that together implement aspects of the temperature synchronization function performed by method 400. Certain aspects of method 400 are also performed by processor assembly 505.

Element 530 is a failsafe circuit that causes thermal elements 210 to shut down if device 100 overheats. For example, if the hot/cold temperature regulation system fails, this fail-safe acts as a back-up or safety feature to prevent a malfunction. In certain embodiments, only the subsystem that is malfunctioning, e.g. the circuitry and controls the provision of hot and/or cold, is shut down; in these embodiments, other features of the device continue to function. In other embodiments, any overheating causes the entire device to shut down.

Element 115 is the magnetic charger interface. Magnetic charger interface 115 appears on the side of device 100. Other types of electric charger interfaces may also be supported in different embodiments, including USBC, and USB.

Elements 540 and 545 are interfaces to the two thermal elements 210.

Elements 550 and 555 are interfaces to the two temperature sensors 305.

### Embodiment with Fan

FIGS. 6-7 illustrate a second embodiment of a skin treatment device that includes a fan for additional ventilation. Generally, this second embodiment has the same principle of operation and has substantially the same design and components and delivers essentially the same features and benefits to a user as the first embodiment, which is described with reference to FIGS. 1-5. Thus, the second embodiment may be considered as describing a variant of device 100. For that reason, unless otherwise specified the operation of device 600 can be assumed to be identical to that of device 100 unless otherwise specified below.

FIG. 6 illustrates a second embodiment of the exterior of a skin treatment device 600 that is used to treat the skin of a user. Similar to device 100, device 600 has two thermal plates 610 that dissipate excess heat or cold generated by the device. As illustrated, each thermal plate 610 has two sets of corresponding vents 650. Vents 650 are used to draw air from the exterior of device 600 into a fan that is disposed below a corresponding thermal plate 650. In other embodiments, there may be one or more than two vents 650 for each thermal plate 650 and their position may be different than as illustrated in FIG. 6.

Device 600 has a housing 605. Housing 605 includes one or more vents 655 that allow warm air from the interior of device 600 to escape. Vents 655 are depicted at the horizontal end of device 600; however, in other embodiments, vents 655 may be disposed in other locations on housing 605. Furthermore, FIG. 6 shows each thermal plate 610 as incorporating two sets of vents 650; however, the number of and placement of vents 655 is a design choice that may vary.

FIG. 7 illustrates a frontal cross-sectional view of device 600. As with device 100, device 600 is right-left symmetric about a central vertical axis B. Many of the elements of device 600 are included on both the right side and the left side; accordingly, the function of only one of such pairs is discussed hereinbelow with the understanding that the comments apply equally to each of the two identical instances. Further, unless otherwise discussed, the form and function of the elements of device 600 are substantially equivalent to their corresponding elements in device 100.

As previously discussed, device 600 has a right and a left thermal plate 610 that dissipates excess heat or cold generated by the device. Although not depicted in FIG. 7, each thermal plate 610 incorporates vents 650. Furthermore, device 600 also includes a fan 760 on both the right and left sides. Fans 760 draws exterior air through vents 650 to increase the ventilation and provide additional cooling as necessary. Exterior air drawn through fans 760 is directed towards and exhausted through vents 655 which are integrated into housing 605, as previously discussed. An air passageway 770 on each side of device 600 enables air to flow between a corresponding fan 760 and vents 655.

Each fan 760 has a small footprint, typically in the range of 20 cm² to 40 cm². In certain embodiments, fan 760 has a single speed and operates at all times, i.e. whenever device 600 is switched on. In other embodiments, fan 760 may be controlled during the operation of device 600; i.e. it may be a variable speed device whose speed is increased or decreased or it may be turned on and off to respond to internal temperature. In certain embodiments, each fan 760 may be independently controlled, e.g., switched on and off, speed increased and decreased. In other embodiments, controls are applied to both fans 760, i.e. they are not independently controlled.

Device 600 further includes a right and a left energy absorption rod 725 whose function is identical to that of absorption rod 225. However, energy absorption rod 725 is somewhat shorter as its vertical endpoint is below fan 760. A heat sink 765 fits on top of energy absorption rod 725 and facilitates the transfer of heat and cold from energy absorption rod 725 to air circulating in air passageway 770 that permits the flow of air between fan 760, energy absorption rod 725 and vents 655.

In operation, when energy absorption rod 725 is conveying heat from thermal element 210 the heat is transferred by heat sink 765 to the air passing through air passageway 770 through vents 655 to the exterior.

Device 600 further includes a printed circuit board (PCB) 655. PCB 655 is substantially the same as PCB 255 with the exception that PCB 655 also supplies power to and controls fan 760.

Generally, the added benefit of device 600 is that enables treatment pads 205 to reach a lower temperature and maintain a lower temperature longer due to the increased air flow provided by fans 760.

Right-Left Temperature Synchronization with Fan

During operation, it is desirable that both right and left treatment pads 205 of device 600 contact a user's skin and provide heating or cooling at approximately the same temperature. It is further desirable that during a cooling treatment both the right and left thermal elements 210, which supply cooling to treatment pads 205, reach a minimum temperature, referred to as Min temp. However, device 600 includes fans 760 which may be activated to provide additional cooling. Thus, in certain cases, fans 760 may be activated to enable one or both of treatment pads 205 to reach Min temp. Therefore, method 450 is slightly modified in device 600 due to the availability of fans 760.

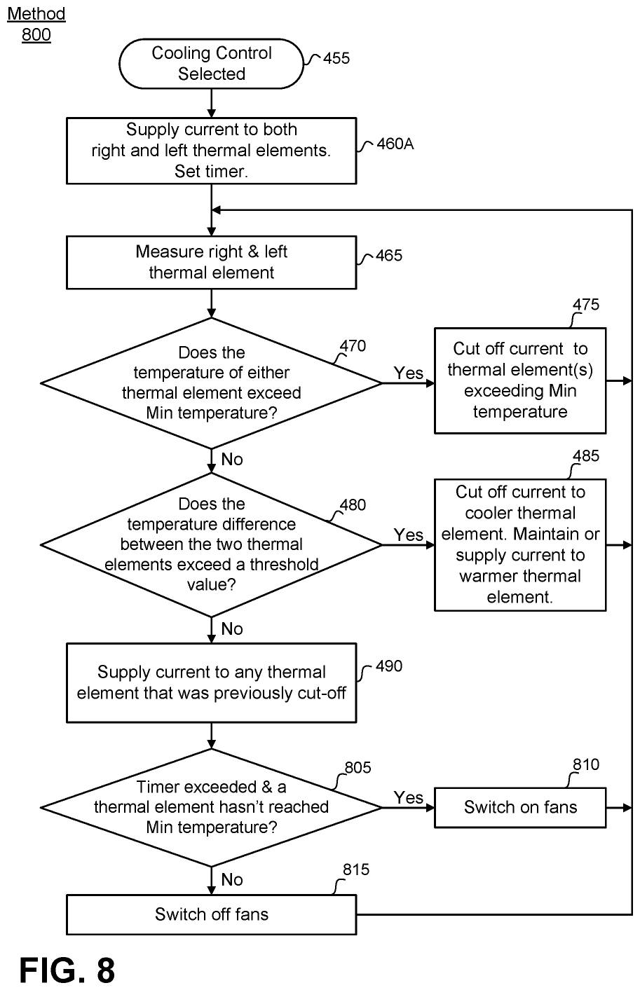

FIG. 8 illustrates one embodiment of a control method 800 that manages the temperatures of two thermal elements 210 during a cooling treatment. The method is identical to method 450 with the exception that if one or both of thermal elements 210 doesn't reach Min temp during a cooling treatment fans 760 are activated. Steps in method 800 that are identical to the corresponding steps method 450 are numbered identically hereinbelow. In method 800, as with method 450, it is assumed that supplying current to a thermal element generates a colder temperature, up to a point where the thermal element does not get colder. Further, the phrase “exceeding a Min temperature” means the temperature is colder than a temperature of Min degrees, where Min is a designated temperature, typically measured in Celsius.

At step 455, method 450 is initiated when a user selects the cooling function of device 100.

At step 460A device 100 supplies a preset current to both thermal elements 210. This current may be a single, designated, current, for example, as prescribed by the manufacturer of thermal element 210. Alternatively, in certain embodiments a variable current may be supplied, which varies over time to achieve a desired level of cooling. Step 460A is identical to step 460 of method 450 except that it additionally sets a timer to measure the time until thermal elements 210 reach min temperature.

At step 465 the temperature of each of the two thermal elements 210 is measured by the corresponding temperature sensor 305.

At step 470 a determination is made as to whether the temperature of either of the two thermal elements 210 exceeds a minimum allowed temperature (Min temperature). This minimum temperature may be established by a medical body or a standards organization or it may be an informal limit.

At step 475 current is cut off to either or both thermal elements 210 whose temperature is determined to exceed the Min temperature. Then processing returns to step 465.

If at step 470 the temperature of neither thermal element 210 exceeds the Min temperature, then processing continues at step 480.

At step 480 an optional determination is made as to whether the temperature difference between the two thermal elements 210 exceed a designated threshold value, e.g. 1 degree C. The temperatures are within the threshold value if their difference is less than the threshold value. If the temperature difference between the two thermal elements 210 exceeds the designated threshold value, then the method continues at step 485. If the difference in temperatures is within the threshold value, then the method continues at step 490.

At step 485 current is cut off to the colder of the two thermal elements 210. If current is flowing to the warmer thermal element 210 the current is maintained. But if no current is flowing to the warmer thermal element 210 then current is turned on to that element. At the conclusion of step 485 processing returns to step 465.

At step 490, if current was previously cut-off to either of the two thermal elements then current is supplied again.

At step 805 a determination is made as to whether the timer as exceeded a preset value and at least one of the two thermal elements 210 hasn't reached the Min temperature. This indicates that additional cooling is required. In which case, at step 810 fans 760 are switched on. If Min temperature has been reached then no additional cooling is required and at step 815 the fans are turned off; or left off, in the case the fans aren't switched on. Then control returns to step 465.

If both fans can be independently operated, then method 800 may be adapted to only switch on a fan 760 that provides additional cooling to a corresponding thermal element 210 that doesn't reach Min temperature within a prescribed interval. Further, if the fan speed of fans 760 may be controlled then additional logic may be implemented to increase or decrease fan speed to achieve a desired temperature.

While the above description of method 800 assumes that current is either supplied or not supplied to a thermal element 210, in other embodiments, current may be increased or decreased rather than being entirely shut off, or rather than being supplied only at a fixed, pre-determined, value.

Upon reading this disclosure, those of skill in the art will appreciate that while particular embodiments and applications have been illustrated and described herein, the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A skin treatment device, comprising:
a housing;
a power source;
a controller, connected to the power source that comprises:
a processor; and
a memory in communication with the processor for storing program instructions and data;
a set of user-selectable controls that enable a user to control the operation of the device;
a right and a left thermal element, each with a bottom side that contacts a treatment pad, wherein during operational use the treatment pad contacts the skin of the user to deliver heating or cooling, or microcurrent; and
wherein the instructions are operable to prevent either of the bottom sides of the right and left thermal elements from exceeding a maximum temperature.

2. The device of claim 1, further comprising:
a red or a blue light, operable to emit red, blue or mixed red and blue light, onto the skin of a user;
wherein the user-selectable controls include a light therapy control, and wherein the light therapy control enables a user to select a setting from among the group consisting of a red light, blue light and purple light, and (1) when the user selects the red light setting a current is supplied to a red light which in turn emits red light, (2) when the user selects blue light a current is supplied to a blue light which in turn emits blue light, and (3) when the user selects purple light a current is supplied to both a red light and a blue light which in turn emits red and blue light.

3. The device of claim 1, wherein the user interface further comprises:
a vibration control.

4. The device of claim 3, further comprising two vibration elements, each in contact with a corresponding treatment pad, wherein when the user activates the vibration control a vibration element causes the corresponding treatment pad to vibrate.

5. The device of claim 1, further comprising:
two temperature sensors, each in proximity to a corresponding thermal element, each of which measure the temperature of the corresponding thermal element at its bottom side and reports the measured temperature to the controller.

6. The device of claim 5, wherein the instructions are further operable to:
receive the reported temperatures from the two temperature sensors;
cut off current to a corresponding thermal element if its bottom side exceeds a maximum temperature, and
cut off current to a corresponding thermal element if its bottom side exceeds a minimum temperature.

7. The device of claim 5, wherein the instructions are further operable to:
maintain the temperature difference between the bottom sides of the right and left thermal elements within a threshold value.

8. The device of claim 7, wherein maintaining the temperature difference between the bottom sides of the right and left thermal elements within a threshold value comprises the steps of:
receiving by the processor the reported temperatures from the two temperature sensors;
determining which of the two bottom sides of the thermal elements is hotter; and
cutting off the current to the hotter of the two thermal elements.

9. The device of claim 5, further comprising
a right fan corresponding to the right thermal element and a left fan corresponding to the left thermal element, wherein each fan enables heat from its corresponding thermal element to be transferred through an air passageway to the exterior of the skin treatment device; and
wherein the user-selectable controls include a cooling control, and
wherein upon detecting that a thermal element doesn't reach the minimum temperature within a designated time interval after the cooling control is selected the right fan and left fan are switched on.

10. The device of claim 1, wherein the right and the left thermal elements are each implemented by a Peltier element.

11. The device of claim 1 further comprising a right and a left absorption rod, wherein the right absorption rod attaches at one end to the right thermal element and at the other end to a right thermal plate on the surface of the device, and the left absorption rod attaches at one end to the left thermal element and at the other end to a left thermal plate on the surface of the device.

12. The device of claim 11 where in the absorption rod is made of a thermally conductive material such as metal that provides thermal conductivity between the thermal element and the thermal plate.

13. The device of claim 1, further comprising:
a right and a left absorption rod, wherein the right absorption rod attaches at one end to the right thermal element and at the other end to a right heat sink, and the left absorption rod attaches at one end to the left thermal element and at the other end to a left heat sink;
a right fan above the right heat sink and a left fan above the left heat sink rod; and
an air passageway between the right heat sink and the right fan and a left air passageway between the left heat sink and the left fan, each air passageway enabling air to flow from the fan to a vent in the housing that enables air to flow to the exterior of the device wherein the air passageway further enables heat transferred by the right and left thermal element to be transferred by a respective absorption rod through the heatsink to the air flowing through the air passageway.

14. The device of claim 13, further comprising:
a right and a left thermal plate on the surface of the device, the right thermal plate disposed above the right fan and the left thermal plate disposed above the left fan and wherein both the left and right thermal plates include one or more vents, wherein during operation of the device, the fan draws air from the exterior into the air passageway.

15. The device of claim 1, wherein the treatment pad is made of a metal such as stainless steel or aluminum.

16. A processor-implemented method performed by a skin treatment device to manage the temperatures of two thermal elements, comprising:
receiving temperature measurements of a bottom side for each of the two thermal elements;
determining if the temperature of the bottom side of either of the two thermal elements exceeds a maximum value;
upon determining that the bottom side of one of the thermal elements exceeds the maximum value cutting off current to the thermal element;
determining if the difference in temperature between the two thermal elements exceeds a threshold value; and
upon determining that the difference in temperature between the two thermal elements exceeds the threshold value cutting off the current to the hotter of the two thermal elements.
